(19) United States
(12) Patent Application Publication
(10) Pub. No.: US 2021/0038845 A1
(43) Pub. Date: Feb. 11, 2021

(54) INTUBATION ASSEMBLY FOR DETERMING WHETHER PATIENT IS BREATHING

(71) Applicants: Si On JO, Seoul (KR); Yuna SEO, Seoul (KR); Si Hyung JO, Seoul (KR); Bum Jin CHANG, Seoul (KR)

(72) Inventors: Si On JO, Seoul (KR); Yuna SEO, Seoul (KR); Si Hyung JO, Seoul (KR); Bum Jin CHANG, Seoul (KR)

(21) Appl. No.: 16/935,545

(22) Filed: Jul. 22, 2020

(30) Foreign Application Priority Data
Aug. 9, 2019 (KR) 10-2019-0097457

(51) Int. Cl. A61M 16/04

(52) U.S. Cl. CPC A61M 16/0488; A61M 16/0475; A61M 16/0402; A61M 2205/6081; A61M 2205/3327; A61M 2205/584; A61M 2205/103

(57) ABSTRACT

Provided is an intubation assembly enabling whether or not a patient is breathing to be determined. The intubation assembly includes an intubation tube and a detector connected to a portion of the intubation tube in an air communicating manner. The detector includes a housing having a transparent window and a movable body disposed within the housing to be movable in response to a flow of air caused by breathing of a patient. The detector attached to a related-art intubation tube enables a doctor or an emergency medical technician to visually or sonically determine whether or not a patient is breathing.

### CROSS REFERENCE TO RELATED APPLICATION

This application claims the benefit under 35 USC 119(a) of Korean Patent Application No. 10-2019-0097457 filed on Aug. 9, 2019 in the Korean Intellectual Property Office, the entire disclosure of which is incorporated herein by reference for all purposes.

### BACKGROUND

### Field

The present disclosure relates to an intubation assembly and, more particularly, to an intubation assembly including an indicator allowing a health care provider, such as a doctor, or emergency medical personnel, such as an emergency medical technician or a first-aider, to visually or sonically determine whether a patient is breathing.

### Description

In general, an intubation tube is a medical instrument intended to maintain the airway of a patient open. Such an operation of maintaining the airway of a patient open is necessarily performed for patients having a respiratory failure or an airway closure, patients whose airway must be safely maintained due to the lack of consciousness, and patients who need manipulation that affects breathing.

Such an intubation tube may be comprised of a tube (i.e. a tracheal tube) serving as a path allowing air to be delivered to the trachea and a connector connecting the tracheal tube to a valve of an Ambu bag or a ventilator, or may be implemented as a combined structure having both the functions of the tracheal tube and the connector.

Such an intubation tube may maintain the airway of a patient open when inserted into the airway of the patient. However, it is extremely difficult to determine whether the tracheal tube is inserted into the airway or the esophagus. That is, the tracheal tube may be inserted into the esophagus instead of the airway when tracheal intubation is performed, and it is difficult to recognize this situation. In a case in which the tracheal tube is inserted into the esophagus of the patient, the breathing of the patient must be severely inhibited. Thus, determination of whether the tracheal tube is inserted into the airway or the esophagus must be performed as promptly as possible after the tracheal intubation is performed.

A related-art intubation tube is disclosed in Korean Patent No. 10-1635443. The intubation tube relates to a medical instrument used to supply air to the airway of a patient. The intubation tube is inserted into the airway of the patient to supply air to the patient during operational procedures including general anesthesia. An airway balloon is fixedly attached to one side of the intubation tube. When the intubation tube is inserted into the airway, the airway balloon is inflated within the airway to press the airway so that the intubation tube is not accidently detached from the airway. A mouth balloon is fixedly attached to the other side of the intubation tube to be inflatable within the mount of the patient. An air injector for supplying air to the mouth balloon is provided. According to the related-art intubation tube, a practitioner may more rapidly and easily administer operational procedures including anesthesia.

In addition, another related-art intubation tube enabling checking the status of a patient is disclosed in Korean Patent Application Publication No. 10-2016-0133139. The intubation tube includes: a tubular body having a leading end portion inserted into the trachea; a balloon-shaped expandable portion surrounding the tubular body; a detection ring surrounding the tubular body; and an expansion tube into which air for expanding the balloon-shaped expandable portion is injected. According to this related art, the detection ring disposed to surround the tubular body allows detection of matter, such as foods or secretions, passing through the tubular body, thereby allowing the status of the patient to be more accurately checked.

However, the above-described related art only proposed the structure of the tube for maintaining the airway open and the method of determining the type and amount of matter passing through the intubation tube. The related art fails to propose any means for determining whether or not the patient is breathing, which is problematic.

Accordingly, there has been increasing demand for the development of a novel and advanced intubation tube assembly, i.e. an intubation assembly, having the shape of an intubation tube maintaining the airway of the patient open, as well as enabling whether or not a patient is breathing to be determined.

The information disclosed in the Background section is only provided for a better understanding of the background and should not be taken as an acknowledgment or any form of suggestion that this information forms prior art that would already be known to a person having ordinary skill in the art.

### BRIEF SUMMARY

The present disclosure has been made keeping in mind the above problems occurring in the related art, and the present disclosure is intended to propose provide an intubation assembly having a detector disposed on a predetermined portion of an intubation tube to determine whether or not a patient is breathing, so that whether a tracheal tube is inserted into the airway or the esophagus is easily determined.

The present disclosure is also intended to provide the detector as a rotatable visual means, by which a doctor or an emergency medical technician (or a first-aider) may easily visually determine whether or not a patient is breathing.

The present disclosure is also intended to provide the detector as a movable visual means, by which a doctor or an emergency medical technician may easily visually determine whether or not a patient is breathing.

The present disclosure is also intended to provide the detector as a visual means able to emit light, so that a doctor or an emergency medical technician may easily visually determine whether or not a patient is breathing.

The present disclosure is also intended to provide the detector as an auditory means able to generate sound, so that the breathing status of a patient may be easily checked.

According to an aspect, provided is an intubation assembly enabling whether or not a patient is breathing to be visually determined. The intubation assembly may include: an intubation tube; and a detector connected to a portion of the intubation tube in an air communicating manner, and including a housing having a transparent window and a movable body disposed within the housing to be movable in response to a flow of air caused by breathing of a patient.

The intubation tube may include a first tube and a second tube separated from the first tube. The housing may have a first tube insertion hole into which one end of the first tube is fixedly inserted and a second tube insertion hole into which one end of the second tube is fixedly inserted.

The movable body may include a propeller rotatably mounted on a shaft fixedly connected to an inside of the housing, the propeller including a plurality of blades.

The propeller may include: a plurality of light-emitting diodes respectively mounted on a portion of a corresponding one of the plurality of blades; and a light-emitting diode controller controlling the plurality of light-emitting diodes to be turned on when the blades rotate.

According to the present disclosure, the intubation assembly allowing whether or not a patient is breathing to be determined provides the following effects.

1) The propeller rotated by the breathing of a patient allows the breathing status of a patient to be visually checked in an easy manner.

2) The light-emitting diode (LED) mounted on the blade of the propeller allows the breathing status of the patient to be easily checked even in a dark place.

3) The movable body comprised of the core and the flutterer allows whether or not the patient is breathing to be visually determined in a different method from the propeller.

4) Due to the sound generator and the sound generating body, whether or not the patient is breathing may be visually determined.

### DESCRIPTION OF DRAWINGS

The above and other objects, features, and advantages of the present disclosure will be more clearly understood from the following detailed description, taken in conjunction with the accompanying drawings, in which:

FIG. 1 is a conceptual view illustrating a basic structure of an intubation assembly according to the present disclosure;

FIG. 2 is a conceptual view illustrating an embodiment in which the movable body is a propeller;

FIG. 3 is a conceptual view illustrating a modified embodiment in which LED devices are mounted on the propeller illustrated in FIG. 2;

FIG. 4 is a conceptual view illustrating a modified embodiment in which a sound generator is mounted on the propeller illustrated in FIG. 2;

FIG. 5 is a conceptual view illustrating an embodiment in which the movable body is a combination of a core and flutterers;

FIG. 6 is a conceptual view illustrating a modified embodiment of the flutterers; and

FIG. 7 is a conceptual view illustrating an embodiment in which the detector is implemented as a sound generator.

### DETAILED DESCRIPTION

Hereinafter, embodiments of the present disclosure will be described in detail with reference to the accompanying drawings. It should be understood that the accompanying drawings may not be drawn to scale, and the same elements may be designated by the same reference numerals, even though they may be used in different drawings.

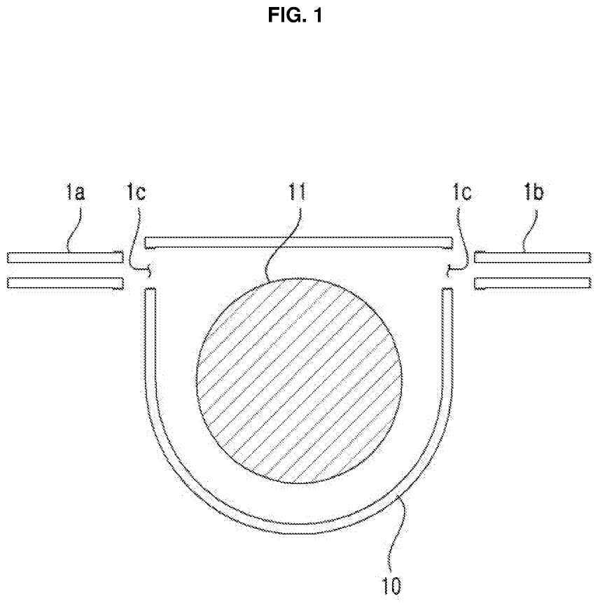

FIG. 1 is a conceptual view illustrating a basic structure of an intubation assembly according to the present disclosure.

As illustrated in FIG. 1, the intubation assembly according to the present disclosure is a structure comprised of an intubation tube 1 and a detector 10.

The intubation tube 1 has a tubular structure allowing air from a person to flow therethrough. The intubation tube 1 is inserted into the airway of a patient in an emergency to maintain the airway of the patient open and allow the patient to breath.

In other words, the intubation tube 1 is configured to be easily bendable according to different oral structures and different airway positions of patients, such that the intubation tube 1 may enter the airway of a patient on an anatomical basis. The intubation tube 1 includes an elastic plastic structure that is bendable and adjustable. In addition, the intubation tube 1 may be made of a biomaterial that is not harmful when in contact with tissues in the human body.

In this manner, the intubation tube 1 serves to help a doctor or an emergency medical technician (or a first-aider) to maintain the airway of a patient open, thereby facilitating a series of following operational procedures including administering general anesthesia. A further description of the structure of the intubation tube 1 will be omitted, since the structure of the intubation tube 1 is similar to that of the above-described intubation tube well-known in the art.

The detector 10 according to the present disclosure is intended to allow the doctor or the emergency medical technician to determine whether or not the patient is breathing when the intubation tube is used. The detector 10 may visually or sonically detect the breathing status of the patient, thereby enabling the doctor or the emergency medical technician to more easily check the breathing status of the patient in an intuitive manner.

In other words, the detector 10 is operated by means of air moved (e.g. a flow of air or a pressure of air caused) by the breathing of the patient to stimulate the sense (e.g. vision or hearing) of the doctor or the emergency medical technician, thereby enabling intuitive detection.

Since the detector 10 operates fundamentally on the basis of air moved by the breathing of the patient, the detector 10 is connected to a portion of the intubation tube 1, exposed out of the body of the patient, so that the air moved by the breathing of the patient may flow to the intubation tube 1.

A specific structure of the detector 10 for providing the visual or auditory detection functions will be described with respect to various following embodiments in conjunction with the accompanying drawings. That is, according to the present disclosure, the detector 10 may include one of a movable body 100 for providing the visual detection function and a sound generator 200 for providing the auditory detection function. Specific structures of the respective components will be described with reference to the accompanying drawings.

The basic structure of the detector 10 includes one of the movable body 100 and the sound generator 200, described above, and a housing 11.

The housing 11 is connected to an external portion of the intubation tube 1 (i.e. a portion exposed outside the human body instead of being inserted into the airway of the patient) so as to communicate with the intubation tube 1. The housing 11 has a hollow structure to provide a physical space in which the movable body or the sound generator to be described later may be accommodated. In particular, in a case in which the detector 10 includes the movable body 100, the housing 11 has a transparent window.

The transparent window is made of a transparent material, and serves to enable the doctor or the emergency medical technician to easily observe the inside of the housing 11. For example, the transparent window may be provided by the entirety of the housing 11 being made of a transparent material or may be provided in a portion of the housing 11 through which the movable body 100 to be described later is exposed. The housing 11 may be integrally connected to the intubation tube 1, or the housing 11 and the intubation tube 1 may be provided separately from each other while being able to be detachably coupled.

Specifically, the intubation tube 1 may be divided into a first tube 1a and a second tube 1b, whereas the housing 11 may have a first tube insertion hole 1c into which one end of the first tube 1a is fixedly fitted and a second tube insertion hole 1c into which one end of the second tube 1b is fixedly fitted.

The first tube 1a is a portion inserted into the body of the patient, and thus, is referred to as an inner tube of the intubation tube, whereas the second tube 1b is a portion exposed outside the body of the patient. The second tube 1b is located opposite to the first tube 1a to extend in a linear structure or another structure.

The housing 11 may have holes to which the ends of the first tube 1a and the second tube 1b are connected. These holes are referred to as the insertion holes 1c.

The insertion holes 1c are two holes such that the ends of the first tube 1a and the second tube 1b may be connected to the two insertion holes 1c, respectively. The insertion holes 1c are illustrated as facing each other in the drawings, although the positions thereof may vary depending on the configurations of the first and second tubes 1a and 1b.

Here, the diameter of each of the insertion holes 1c is determined to be insignificantly greater than the diameter of the intubation tube 1, and packings are provided on the inner circumferential surfaces of the insertion holes 1c, thereby ensuring the first tube 1a and the second tube 1b are firmly connected to the two insertion holes 1c in an air tight manner.

Hereinafter, various structures of the detector 10 will be described.

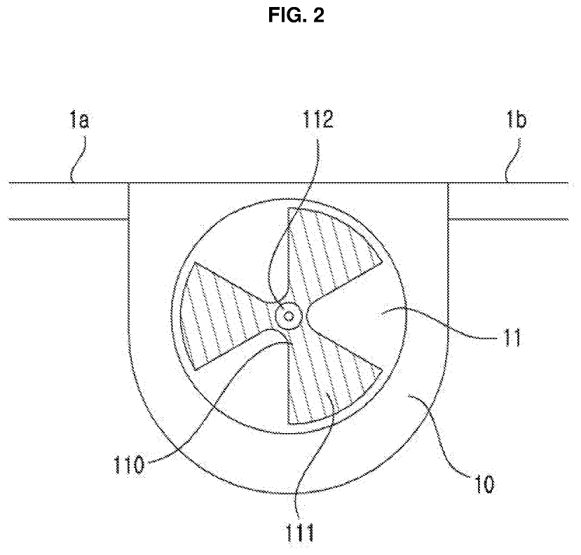

FIG. 2 is a conceptual view illustrating an embodiment in which the movable body is a propeller.

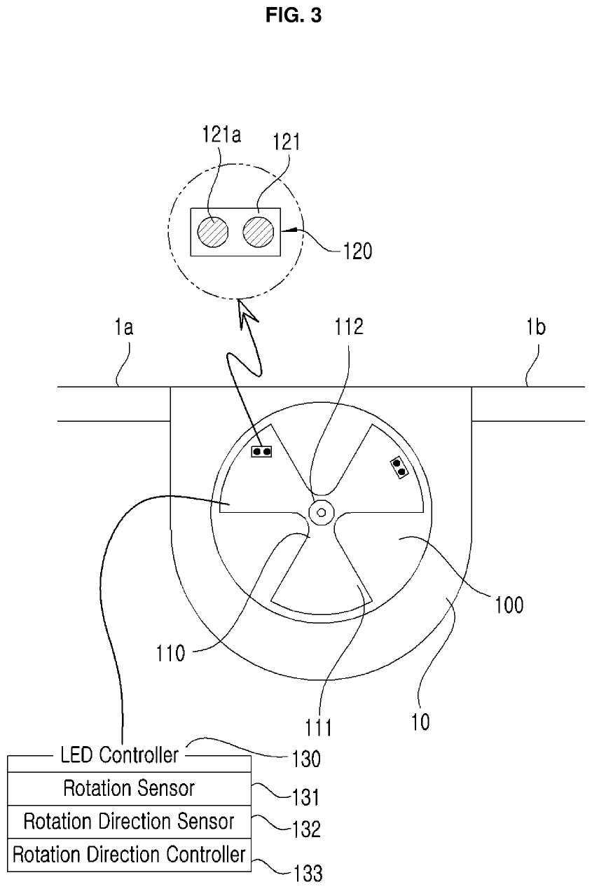

Embodiments illustrated in FIGS. 2 and 3 relate to structures in each of which the detector 10 includes the movable body 100.

First, in the embodiment illustrated in FIG. 2, the movable body 100 operates by means of a propeller 110. Here, the detector 10 includes the housing 11 and the movable body 100 including the propeller 110.

The movable body 100 is disposed within a hollow space of the housing 11. The movable body 100 is movable by a pressure of air caused by the breathing of the patient, thereby enabling the doctor or the emergency medical technician to determine whether or not the patient is breathing.

As illustrated in FIG. 2, the inside of the housing 11 has a hollow structure able to accommodate the movable body 100 implemented as the propeller 110.

That is, in the embodiment illustrated in FIG. 2, the movable body 100 is implemented as the propeller 110 configured such that blades 111 rotate about a rotary shaft 112 located within the housing 11.

Here, air moved by the breathing of the patient needs to arrive at the distal ends of the blades 111 disposed around the rotary shaft 112, thereby causing the blades 111 to rotate. In this regard, the rotary shaft 112 may be positioned in a portion of the housing 11 spaced apart a predetermined distance from a virtual straight line extending within the housing 11, along a flow of air caused by the breathing of the patient.

In addition, in this position, the rotary shaft 112 extends in a direction perpendicular to the above-described straight line so that air comes into contact with a side surface of each of the blades 111, thereby causing the blades 111 to rotate in a direction in which the straight line extends, i.e. a direction of the flow of air.

The blades 111 may have a variety of shapes, such as an elliptical shape, a rod-like shape, or a twisted shape.

Here, the blades 111 may be a plurality of blades. Although the blades 111 may be configured such that the distances from the center of the rotary shaft 112 to the distal ends of the blades 111 are the same or different, the distances from the center of the rotary shaft 112 to the distal ends of the blades 111 may be constant such that the blades 111 may be smoothly rotated by the air moved by the breathing of the patient.

In addition, the propeller 110 may include a luminous material provided on the blades 111 or the like to generate colored light. Consequently, in case of an emergency patient and even in a dark night or in a place with limited or insufficient light, instead of in an operating room or in a place with insufficient light, the doctor or the emergency medical technician may easily visually determine whether or not the patient is breathing by detecting the color of light of the propeller 110.

According to this structure, the propeller 110 rotates in the direction of the flow of air inhaled and exhaled along the intubation tube 1 in response to the breathing of the patient. In this case, the propeller 110 may rotate in a forward direction or a reverse direction in response to the inhalation or exhalation of the patient. Accordingly, the doctor or the emergency medical technician may easily check the breathing status of the patient by visually determining the forward or reverse rotation of the propeller 110.

FIG. 3 is a conceptual view illustrating a modified embodiment in which LED devices are mounted on the propeller illustrated in FIG. 2.

The embodiment illustrated in FIG. 3 relates to a structure in which light-emitting diode (LED) devices 120 are mounted on the blades 111 of the propeller 110 described above with reference to FIG. 2.

The LED devices 120 serve to directly convert electron energy into light energy. Differently from a majority of the other light sources, such as fluorescent lamps or incandescent lamps, the LED devices 120 may produce light without ultraviolet (UV) radiation or infrared (IR) radiation in a simple manner. According to the present disclosure, the LED devices 120 may be provided as not only individual LEDs 121a but also as LED packages 121 in each of which a plurality of LEDs 121a are grouped.

The LED devices 120 may be mounted on the two or more of the blades 111 of the propeller 110, or may also be mounted on a single one of the blades 111 of the propeller 110. In addition, a single one of the blades 111 may have a plurality of LED devices 120 or a single LED device 120 mounted thereon.

An LED controller 130 serves to control the LED devices 120 to be turned on or off. The LED controller 130 is implemented as a circuit able to turn on or off the LED devices 120 by receiving power from a power supply provided in one portion of the housing 11.

Here, the LED controller 130 may include a rotation sensor 131.

The rotation sensor 131 serves to convert physical signals generated during the rotation of the blades 111 into electrical signals for the turning on or off of the LED devices 120. The rotation sensor 131 may have a variety of structures well-known in the art. For example, the rotation sensor 131 including a limit switch and a terminal will be described.

The terminal protrudes from one portion of the circumferential surface of the rotary shaft 112, and the limit switch is provided on one portion of the housing 11 at the periphery of the rotary shaft 112. The limit switch comes into contact with the terminal to obtain electrical signals, in response to the rotation of the rotary shaft 112. In other words, the limit switch serves to generate electrical signals to turn on and off the LED devices 120 by detecting whether or the rotary shaft 112 rotates.

According to this structure, in case of an emergency patient and even in a dark night or in a place with limited or insufficient light, instead of in an operating room or in a place with insufficient light, the doctor or the emergency medical technician may easily visually determine whether or not the patient is breathing by detecting the turning on or off of the LED devices 120 mounted on the blades 111 of the propeller 110.

In addition, the LED controller 130 may include a rotation direction sensor 132 detecting the direction of rotation of the blades 111.

The rotation direction sensor 132 may be implemented as a well-known direction sensor, such as a gyro sensor or an acceleration sensor, detecting rotational inertia using acceleration information of the blades 111 occurring during the rotation of the blades 111. Hereinafter, an embodiment realized by modifying the above-described limit switch will be described as an example.

The limit switch according to the present embodiment may be a plurality of limit switches differently from the limit switch according to the foregoing embodiment. Specifically, the plurality of limit switches may be provided in order along the circumference of the housing 11 at the periphery of the rotary shaft 112 and spaced apart from each other predetermined distances.

That is, the direction of rotation of the blades 111 may be detected according to the order in which the plurality of limit switches come into contact with the terminal. For example, during the rotation of the blades 111, when the order in which the plurality of limit switches come into contact with the terminal is 1, 2, and 3, the rotation direction sensor 132 that has received signals from the limit switches of the blades 111 detects the direction of rotation to be clockwise. When the order in which the plurality of limit switches come into contact with the terminal is 3, 2, and 1, the rotation direction sensor 132 detects the direction of rotation to be counterclockwise. In other words, the rotation direction sensor 132 based on the plurality of limit switches may detect the direction of the rotation of the blades 111 by detecting the order in which the terminal comes into contact with the plurality of limit switches.

In a corresponding manner, the LED controller 130 includes a rotation direction controller 133 differentially controlling the colors of the LED devices 120 according to the direction of rotation of the blades 111.

For example, in case in which the LED devices 120 are the LED packages 121 respectively including a first LED 121a emitting red light and a second LED 121a emitting green light, the rotation direction controller 133 controls the first LEDs 121a to be turned on when receiving a signal notifying that the blades 111 have rotated in the clockwise direction from the rotation direction sensor 132 and controls the second LEDs 121a to be turned on when receiving a signal notifying that the blades 111 have rotated in the counterclockwise direction from the rotation direction sensor 132. In a case in which the blades 111 rotating in the clockwise direction corresponds to the inhalation of the patient and the blades 111 rotating in the counterclockwise direction corresponds to the exhalation of the patient, the doctor or the emergency medical technician may easily determine whether or not the patient is repeatedly inhaling and exhaling by detecting the colors of the LED devices 120.

According to this structure, the doctor or the emergency medical technician may rapidly treat the emergency patient by precisely checking the breathing status of the patient with unbalanced inhalation and exhalation by detecting or controlling the direction of rotation of the blades 111.

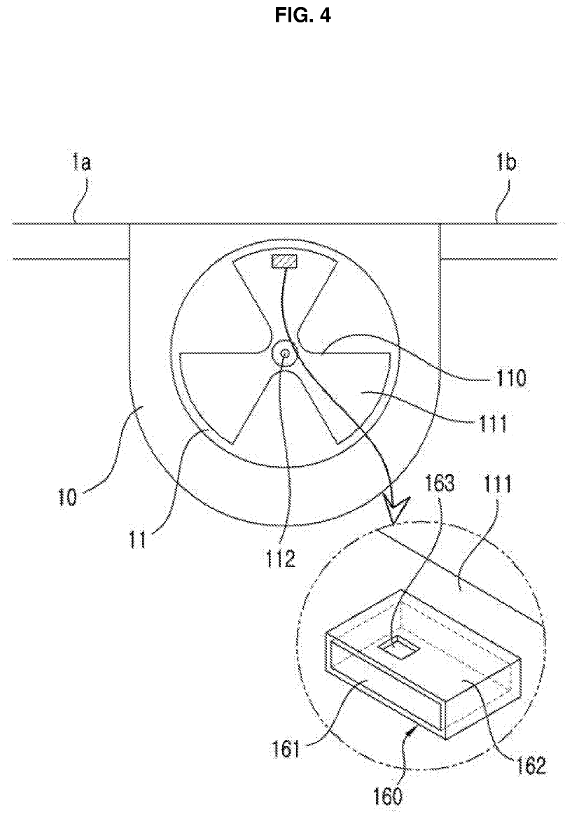

FIG. 4 is a conceptual view illustrating a modified embodiment in which a sound generator is mounted on the propeller illustrated in FIG. 2.

The intubation assembly according to the embodiment illustrated in FIG. 4 includes a sound generator 160 mounted on at least one of the blades 111 to generate sound when the blades 111 rotate.

The sound generator 160 is mounted on at least one of the plurality of blades 111, and serves to enable the doctor or the emergency medical technician to determine whether or not the patient is breathing by auditory detection.

The sound generator 160 may not only be integrally provided on a portion of the blades 111 but also be mounted on the blades 111 as a structural portion thereof.

In addition, the sound generator 160 may be a plurality of sound generators mounted on a single one of the blades 111. Alternatively, a plurality of sound generators 160 may be mounted on each of the plurality of blades 111, or a single sound generator 160 may be mounted on each of the plurality of blades 111.

Specifically, the sound generator 160 includes an air inlet 161, a space 162, and an air outlet 163.

The air inlet 161 is a hole through which air caused by the breathing of the patient enters the inside of the blade 111. The space 162 is a hollow space in which the air entered through the air inlet 161 is temporarily stored.

The air outlet 163 is a hole through the air is discharged, and the diameter of the air outlet 163 is smaller than the diameter of the air inlet 161. Since the diameter of the air outlet 163 is smaller than the diameter of the air inlet 161, a sound wave may be generated by a difference in pressure when the air entered the space 162 through the air inlet 161 exits through the air outlet 163 having the smaller diameter, i.e. the narrower opening.

A portion of the air entered through the air inlet 161 exits through the air outlet 163, and the remaining portion of the air remains in the space 162. After the portion of the air has completely exited, the remaining portion of the air exits through the air outlet 163. When a direction in which the air enters is substantially perpendicular to a direction in which the air exits, the sound wave may be generated. The sound wave may be amplified as the remaining portion of the air that has remained in the space 162 exits. That is, more preferably, the air inlet 161 and the air outlet 163 may be directed perpendicular to each other. In a corresponding manner, a hole, i.e. a sound output hole, allowing the sound wave to be output therethrough may be provided in a portion of the housing 11.

Accordingly, as the air caused by the breathing of the patient generates the sound wave while entering and exiting the sound generator 160, the doctor or the emergency medical technician may sonically detect the sound wave, thereby determining whether or not the patient is breathing. In addition, the auditory detection function may be added to the visual detection function based on the rotation of the propeller 110.

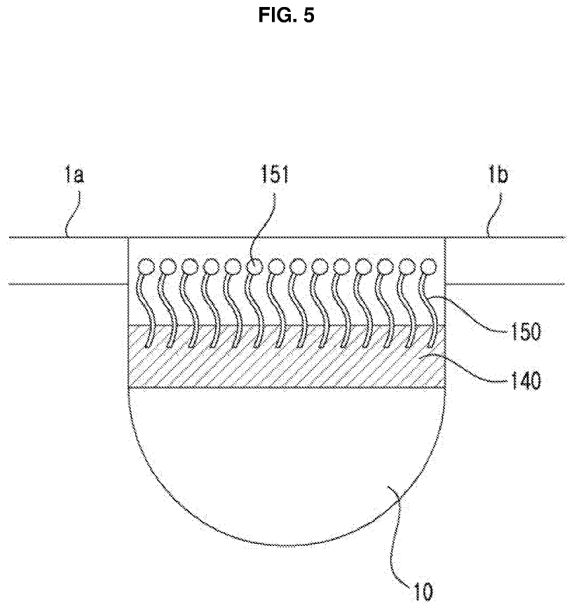

FIG. 5 is a conceptual view illustrating an embodiment in which the movable body is a combination of a core and flutterers.

In the embodiment illustrated in FIG. 5, the movable body 100 operates by means of a core 140 and flutterers 150.

The core 140 is a three-dimensional structure extending by a predetermined distance and located within the housing 11. The core 140 may extend in a variety of directions within the housing 11.

For example, the core 140 may extend by a predetermined length within the housing 11, not only in the direction in which the air caused by the breathing of the patient flows but also in the direction perpendicular to the direction in which the air flows. Here, the core 140 may extend in the same direction as the direction in which the air flows in order to maximize the fluttering (e.g. waving or flapping) of the flutterers 150, since the flutterers 150 to be described later generally extend from the surface of the core 140 in a direction perpendicular to a direction in which the core 140 extends.

Here, the core 140 may be disposed within the housing 11 so as to be rotatable by means of a connecting shaft.

The flutterers 150 are a plurality of structures similar to bristles of a brush, extending from the surface of the core 140 and spaced apart from each other predetermined distances. The flutterers 150 are configured to wave in the wind, like bristles of a duster.

The flutterers 150 may be made of a light material, since the flutterers 150 must wave in the air caused by the breathing of the patient. For example, the flutterers 150 may be made of a light material, such as nylon or fiber.

According to this structure, the flutterers 150 mounted on the core 140 are movable or flexible to wave in the air caused by the breathing of the patient.

Accordingly, the doctor or the emergency medical technician may visually determine whether or not the patient is breathing, on the basis of the movement of the flutterers 150, by watching the flutterers 150 mounted on the core 140 of the movable body 100.

In addition, the core 140 and the flutterers 150 may include a luminous material able to generate colored light. In case of an emergency patient and even in a dark night or in a place with limited or insufficient light, instead of in an operating room or in a place with insufficient light, the doctor or the emergency medical technician may easily visually determine whether or not the patient is breathing by detecting the color of light generated by the luminous material of the core 140 or the flutterers 150.

In addition, weights 151 may be mounted on the distal ends of the flutterers 150, respectively. The weights 151 have greater diameters than the distal ends of the flutterers 150, and have a color different from that of the distal ends of the flutterers 150.

A plurality of weights 151 may be mounted on each of the plurality of flutterers 150, or a single weight 151 may be mounted on each of the plurality of flutterers 150.

When the plurality of weights 151 are mounted on each of the plurality of flutterers 150, the weights 151 may have different colors, such as a luminous color, a fluorescent color, or different shapes.

The weights 151 having greater diameters than the distal ends of the flutterers 150 are intended to prevent the flutterers 150 from excessively waving, impart suitable masses to the flutterers 150, and catch the attention of the doctor or the emergency medical technician.

According to this structure, the doctor or the emergency medical technician may more easily determine whether or not the patient is breathing by watching the weights 151 mounted on the distal ends of the flutterers 150 than by simply watching the flutterers 150.

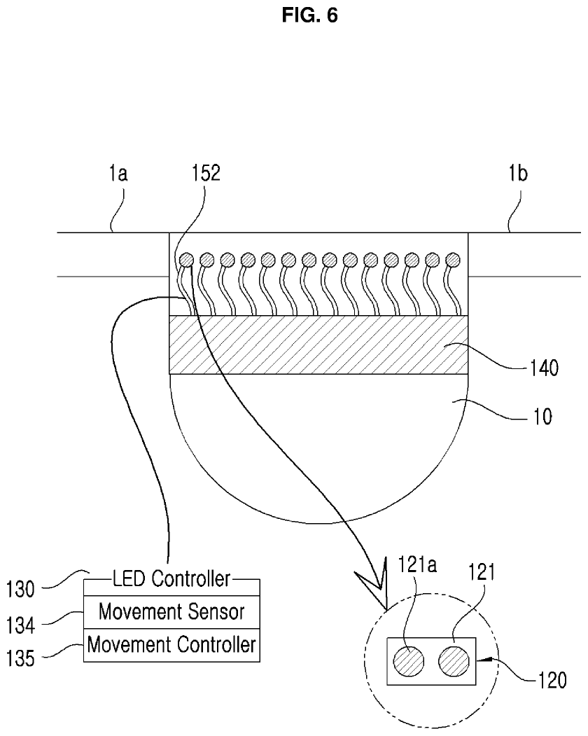

FIG. 6 is a conceptual view illustrating a modified embodiment of the flutterers.

In the embodiment illustrated in FIG. 6, each of the flutterers 150 illustrated in FIG. 5 includes a cable 152 and an LED device 120.

Each of the flutterers 150 illustrated in FIG. 5 includes the cable 152 and the LED device 120.

The cable 152 extends from the surface of the core by a predetermined distance and is spaced apart from adjacent cables 152 predetermined distances. The cable 152 is comprised of two or more wires or optical fibers bound together to supply electricity to the corresponding LED device 120. The cable 152 may be a plurality of cables provided on the surface of core 140, or may be provided as a single cable. That is, the cable 152 not only serves to supply power to the LED device 120 to be described later, but also is movable by the flow of the air like the flutterers 150 described above with reference to FIG. 5, thereby providing the visual detection function.

The LED device 120 is disposed on the distal end of the cable 152. The LED device 120 may be a plurality of LED devices or a single LED device connected to each of a plurality of cables. Alternatively, a plurality of LED devices or a single LED device may be mounted on the distal end of a single cable.

Here, a movement sensor 134 detecting the movement of the cable 152 is provided on each of the flutterers 150.

The movement sensor 134 serves to convert physical signals generated during the movement of the cable 152 into electrical signals for the turning on or off of the LED device 120. The movement sensor 134 may have a variety of structures well-known in the art. For example, the movement sensor 134 may be implemented as a gyro sensor detecting the movement of the cable 152.

The movement sensor 134 based on the gyro sensor serves to the inertia of the cable 152 when the cable 152 moves. The movement sensor 134 may be a plurality of movement sensors or a single movement sensor provided on a portion of the cable 152.

In addition, the movable body illustrated in FIG. 6 including the flutterers 150 includes an LED controller 130. The LED controller 130 further includes a movement controller 135 controlling the LED device 120 to be turned on or off when the cable 152 moves, in addition to the movement sensor 134.

When the cable 152 moves in contact with the air caused by the breathing of the patient, the movement controller 135 serves to turn on the LED device 120 by receiving a movement detection signal from the movement sensor 134. Accordingly, the movement controller 135 may turn on the LED device 120 when the cable 152 moves and turn off the LED device 120 when the cable 152 does not move, thereby turning on the LED device 120 only when the patient breathes.

According to this structure, the turning on of the LED device 120 mounted on the flutterer 150 may enable the doctor or the emergency medical technician to easily visually determine whether or not the patient is breathing, even in a place with limited or insufficient light.

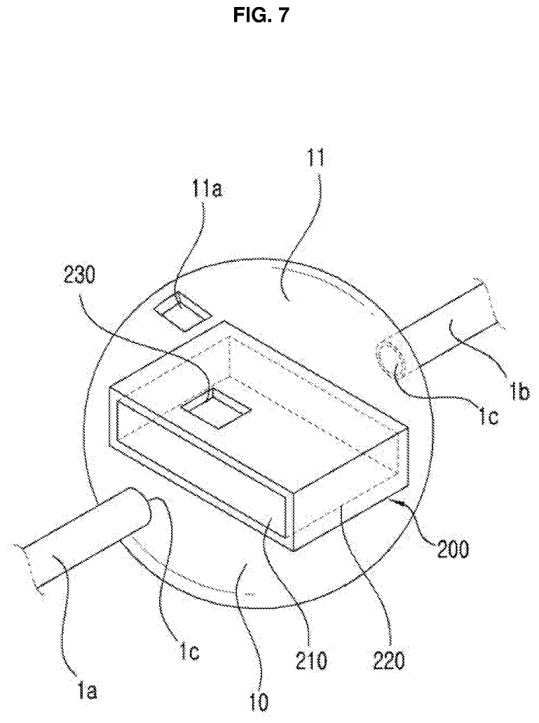

FIG. 7 is a conceptual view illustrating an embodiment in which the detector is implemented as a sound generator.

While the detector 10 in the foregoing embodiment has been described as providing the visual detection function or the auditory detection function, the embodiment illustrated in FIG. 7 relates to a structure in which the detector 10 only provides the auditory detection function without providing the visual detection function.

Specifically, the detector 10 illustrated in FIG. 7 may include the housing 11 and the sound generator 200 respectively having the above-described structure or only include the sound generator 200 without the housing 11.

The sound generator 200 is disposed within the housing 11 or provided on an externally-exposed portion of the intubation tube 1 to generate sound by a pressure of air caused by the breathing of the patient. In FIG. 7, the embodiment in which the sound generator 200 is disposed within the housing 11 is illustrated. The structure of sound generator 200 is similar to or the same as the structure of sound generator 160 described above.

Here, the housing 11 has a plurality of through-holes 11a or a single through-hole 11a. This configuration is intended to help sound generated by the sound generator 200 to exit the housing 11, so that the sound may be more efficiently transmitted to the doctor or the emergency medical technician.

Here, the sound generator 200 includes an air inlet 210, a space 220, and the air outlet 230, like the above-described sound generator 160.

Like the sound generator 160, the sound generator 200 may be configured such that a direction in which the air enters through the air inlet 210 and a direction in which the air exits through the air outlet 230 are substantially perpendicular to each other, so as to amplify the sound, thereby improving the transmission of the sound to the doctor or the emergency medical technician.

Since the structure and functions of the sound generator 200 are similar to those of the sound generator 160 described above, detailed descriptions of the structure and functions of the sound generator 200 will be omitted.

According to the above-described structure, a doctor or an emergency medical technician may easily sonically determine whether or not a patient is breathing by detecting sound generated by the sound generator 200.

The configurations and functions of the intubation assembly according to the present disclosure have been described with reference to the drawings. It should be understood, however, that the foregoing descriptions are illustrative only, and the technical idea of the present disclosure is not limited to the foregoing descriptions or the accompanying drawings. Those having ordinary knowledge in the art will appreciate that various modifications and changes in forms are possible without departing from technical idea of the present disclosure.

What is claimed is:

1. An intubation assembly enabling whether or not a patient is breathing to be visually determined, the intubation assembly comprising:
an intubation tube; and
a detector connected to a portion of the intubation tube in an air communicating manner, and comprising a housing having a transparent window and a movable body disposed within the housing to be movable in response to a flow of air caused by breathing of a patient.

2. The intubation assembly according to claim 1, wherein the intubation tube comprises a first tube and a second tube separated from the first tube,
wherein the housing has a first tube insertion hole into which one end of the first tube is fixedly inserted and a second tube insertion hole into which one end of the second tube is fixedly inserted.

3. The intubation assembly according to claim 1, wherein the movable body comprises a propeller rotatably mounted on a shaft fixedly connected to an inside of the housing, the propeller including a plurality of blades.

4. The intubation assembly according to claim 3, wherein the propeller comprises:
a plurality of light-emitting diodes respectively mounted on a portion of a corresponding one of the plurality of blades; and
a light-emitting diode controller controlling the plurality of light-emitting diodes to be turned on when the blades rotate.

5. The intubation assembly according to claim 4, wherein the light-emitting diode controller comprises:
a rotation direction sensor detecting a direction of rotation of the blades; and
a rotation direction controller differentially controlling colors of the light-emitting diodes depending on the direction of rotation of the blades.

6. The intubation assembly according to claim 3, wherein at least one of the plurality of blades of the propeller comprises a sound generator, the sound generator including:
an air inlet through which the air enters;
a space in which the air entered through the air inlet is temporarily stored; and
an air outlet through which the air exits, the air outlet having a smaller diameter than the air inlet.

7. The intubation assembly according to claim 1, wherein the movable body includes:
a three-dimensional core extending by a predetermined distance and located within the housing; and
a plurality of flutterers extending by predetermined distances from a surface of the core and being spaced apart from each other predetermined distances, the plurality of flutterers being movable in response to a pressure of the flow of air.

8. The intubation assembly according to claim 7, wherein each of the flutterers comprises:
a cable through which electricity is supplied to a light-emitting diode, wherein the cable extends from a surface of the core by a predetermined distance and is spaced apart from adjacent cables predetermined distances;
the light-emitting diode being mounted on a distal end of the cable; and
a movement sensor detecting the cable being movable in response to the pressure of the air, and
wherein the movable body comprises a light-emitting diode controller including a movement controller controlling the light-emitting diode to be turned on when the cable moves.

9. The intubation assembly according to claim 7, wherein each of the flutterers comprises a weight mounted thereon, the weight having a greater diameter than a distal end of the flutterer and having a different color from the distal end of the flutterer.

10. An intubation assembly enabling whether or not a patient is breathing to be sonically determined, the intubation assembly comprising:
an intubation tube; and
a detector connected to a portion of the intubation tube in an air communicating manner, and comprising a sound generator generating sound in response to a pressure of air caused by breathing of a patient.

11. The intubation assembly according to claim 10, wherein the sound generator includes:
an air inlet through which the air enters;
a space in which the air entered through the air inlet is temporarily stored; and
an air outlet through which the air exits, the air outlet having a smaller diameter than the air inlet.
